(19) United States
(12) Patent Application Publication
(10) Pub. No.: US 2023/0309965 A1
(43) Pub. Date: Oct. 5, 2023

(54) SUBSTRATE CONNECTION STRUCTURE, ULTRASOUND DIAGNOSIS APPARATUS AND ULTRASOUND ENDOSCOPE

(71) Applicant: OLYMPUS CORPORATION, Tokyo (JP)

(72) Inventor: Sunao SATO, Yamato-shi (JP)

(73) Assignee: OLYMPUS CORPORATION, Tokyo (JP)

(21) Appl. No.: 18/207,015

(22) Filed: Jun. 7, 2023

Related U.S. Application Data
(63) Continuation of application No. PCT/JP2020/048496, filed on Dec. 24, 2020.

(51) Int. Cl. A61B 8/00; A61B 8/12

(52) U.S. Cl. CPC A61B 8/4488; A61B 8/12; A61B 1/018

(57) ABSTRACT

A substrate connection structure includes: a rigid substrate that includes a plurality of electrodes and a plurality of wiring patterns that are electrically connected to the plurality of electrodes; a flexible printed board that includes a plurality of flying leads formed on one end of the flexible printed board, the flying leads being electrically connected to the plurality of electrodes; and second protrusions that are arranged on the plurality of electrodes and on which the flying leads are arranged.

### CROSS-REFERENCE TO RELATED APPLICATION

This application is a continuation of International Application No. PCT/JP2020/048496, filed on Dec. 24, 2020, the entire contents of which are incorporated herein by reference.

### BACKGROUND

### 1. Technical Field

The present disclosure relates to a substrate connection structure between a flexible printed board including flying leads and a rigid substrate, an ultrasound diagnosis apparatus (an ultrasound imaging apparatus) and an ultrasound endoscope.

### 2. Related Art

In the related art, an ultrasound diagnosis apparatus that transmits ultrasound waves to a subject, such as a human body, receives echo signals that are reflected by living tissues, and obtains tomographic images is widely used in the field of diagnostic medical care. In the ultrasound diagnosis apparatus, a flexible printed board (hereinafter, abbreviated as an “FPC board”) in which a plurality of ultrasound transducers and a plurality of cables that transmit and receive signals are connected is electrically connected to a rigid substrate that is electrically connected to the ultrasound transducers.

If the soft FPC board and the rigid substrate are fusion-spliced by heating solder or the like, electrically connected portions may be deviated from each other due to a difference between thermal expansion coefficients of the FPC board and the rigid substrate. As a technology for preventing the deviation between the electrically connected portions, a connection method has been proposed in which a connection portion of the FPC board with respect to the rigid substrate is formed as a flying lead (for example, see Japanese Patent No. 3802756).

### SUMMARY

In some embodiments, a substrate connection structure includes: a rigid substrate that includes a plurality of electrodes, and a plurality of wiring patterns that are electrically connected to the plurality of electrodes; a flexible printed board that includes a plurality of flying leads formed on one end of the flexible printed board, the flying leads being electrically connected to the plurality of electrodes; and second protrusions that are arranged on the plurality of electrodes and on which the flying leads are arranged.

In some embodiments, an ultrasound diagnosis apparatus includes: a plurality of ultrasound transducers each transmitting and receiving ultrasound waves; a rigid substrate that includes a plurality of electrodes, and a plurality of wiring patterns that are electrically connected to the plurality of electrodes and the plurality of ultrasound transducers, the rigid substrate being fixed to the plurality of ultrasound transducers; a flexible printed board that includes a plurality of flying leads formed on one end of the flexible printed board, the flying leads being electrically connected to the plurality of electrodes; and second protrusions that are arranged on the plurality of electrodes and on which the flying leads are arranged.

In some embodiments, an ultrasound endoscope includes: an insertion portion configured to be inserted into a subject; and an ultrasound transducer unit arranged on the insertion portion, the ultrasound transducer unit including: a plurality of ultrasound transducers each transmitting and receiving ultrasound waves; a rigid substrate that includes a plurality of electrodes, and a plurality of wiring patterns that are electrically connected to the plurality of electrodes and the plurality of ultrasound transducers, the rigid substrate being fixed to the plurality of ultrasound transducers; a flexible printed board that includes a plurality of flying leads formed on one end of the flexible printed board, the flying leads being electrically connected to the plurality of electrodes; and second protrusions that are arranged on the plurality of electrodes and on which the flying leads are arranged.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is an overall configuration diagram of an ultrasound diagnosis apparatus according to one embodiment of the disclosure;

FIG. 2 is a diagram illustrating a configuration of a distal end portion of an insertion portion of the ultrasound diagnosis apparatus illustrated in FIG. 1;

FIG. 3 is a cross-sectional view of a transducer unit in a longitudinal direction, which is used in the ultrasound diagnosis apparatus illustrated in FIG. 1;

FIG. 4 is a plan view for explaining a transducer substrate;

FIG. 5 is a plan view for explaining a cable substrate;

FIG. 6A is a partially enlarged plan view of connection portions of the transducer substrate and the cable substrate;

FIG. 6B is a partially enlarged side view of the connection portions of the transducer substrate and the cable substrate;

FIG. 7 is a partially enlarged plan view of connection portions of a transducer substrate and a cable substrate according to a first modification of the embodiment of the disclosure;

FIG. 8 is a partially enlarged plan view of connection portions of a transducer substrate and a cable substrate according to a second modification of the embodiment of the disclosure;

FIG. 9A is a partially enlarged plan view of connection portions of a transducer substrate and a cable substrate according to a third modification of the embodiment of the disclosure; and

FIG. 9B is a partially enlarged side view of the connection portions of the transducer substrate and the cable substrate according to the third modification of the embodiment of the disclosure.

### DETAILED DESCRIPTION

As modes for carrying out the disclosure (hereinafter, referred to as “embodiments”), an ultrasound diagnosis apparatus will be described below. The disclosure is not limited by the embodiment below. Further, in description of the drawings, the same components are denoted by the same reference symbols. Furthermore, it is necessary to note that the drawings are schematic, and relationships between thicknesses and widths among the components, ratios among the components, and the like may be different from the actual ones. Moreover, the drawings may include a portion that has different dimensional relations or ratios.

### Embodiment

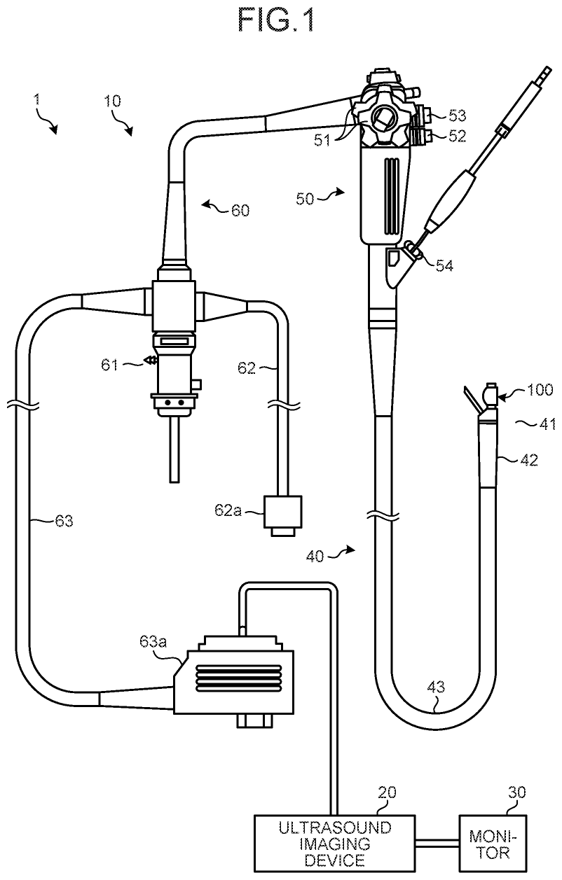

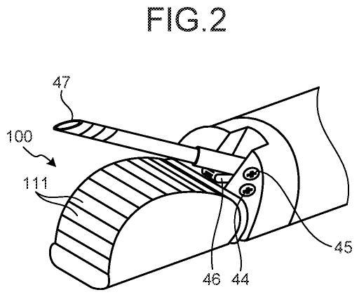

FIG. 1 is an overall configuration diagram of an ultrasound diagnosis apparatus according to one embodiment of the disclosure. FIG. 2 is a diagram illustrating a configuration of a distal end portion of an insertion portion of the ultrasound diagnosis apparatus illustrated in FIG. 1. An ultrasound diagnosis apparatus 1 includes an ultrasound endoscope 10, an ultrasound imaging device 20, and a monitor 30. Further, the ultrasound endoscope 10 includes a thin and elongated insertion portion 40 that is inserted into a body, an operating unit 50 that is connected to a proximal end of the insertion portion 40, and a universal cord 60 that is extended from a side portion of the operating unit 50.

A connector 61 that is connected to a light source device (not illustrated) is arranged on a proximal end portion of the universal cord 60. A cable 62 that is connected to a camera control unit (not illustrated) via a connector 62a and a cable 63 that is detachably connected to the ultrasound imaging device 20 via a connector 63a are extended from the connector 61. Further, the ultrasound imaging device 20 is connected to the ultrasound endoscope 10 via the connector 63a, and the monitor 30 is also connected to the ultrasound endoscope 10 via the ultrasound imaging device 20.

A main part of the insertion portion 40 is configured by connecting a distal end rigid portion (hereinafter, referred to as a “distal end portion”) 41, a bending portion 42 that is located at a rear end of the distal end portion 41, and a flexible tube portion 43 that is located at a rear end of the bending portion 42, that has a small diameter, that is elongated, that has flexibility, and that is elongated to the operating unit 50, in this order from a distal end side.

A transducer unit 100 is arranged on a distal end side of the distal end portion 41. In the distal end portion 41, an illumination lens 44 that constitutes an illumination optical system, an observation lens 45 of an observation optical system, an forceps opening (not illustrated) as a distal end opening that also functions as a suction opening are arranged at the side of a base portion relative to the transducer unit 100. The forceps opening is an outlet of a treatment tool insertion path. A treatment tool raising board 46 is arranged in the forceps opening. An operation wire (not illustrated) is connected to the treatment tool raising board 46, and, by operating a forceps raising knob (not illustrated), it is possible to pull the operation wire and adjust a drawing angle of a puncture needle 47 that is drawn out of the treatment tool insertion path.

In the operating unit 50, angle knobs 51 that cause the bending portion 42 to bend in a desired direction, an air-supply and water-supply button 52 for performing air supply operation and water supply operation, a suction button 53 for performing suction operation, and a treatment tool insertion opening 54 that serves as an entrance for a treatment tool that is introduced into a body are arranged.

The treatment tool insertion opening 54 is connected to the forceps opening via a treatment tool insertion channel (not illustrated) that is arranged inside the insertion portion 40. The treatment tool insertion opening 54 allows insertion of a sheath of an ultrasound treatment tool (not illustrated). Further, by causing the puncture needle 47 that is inserted in the sheath to protrude from the forceps opening, it is possible to arrange the puncture needle 47 in an observation field of the transducer unit 100 such that the puncture needle 47 can move in both of a forward direction and a backward direction.

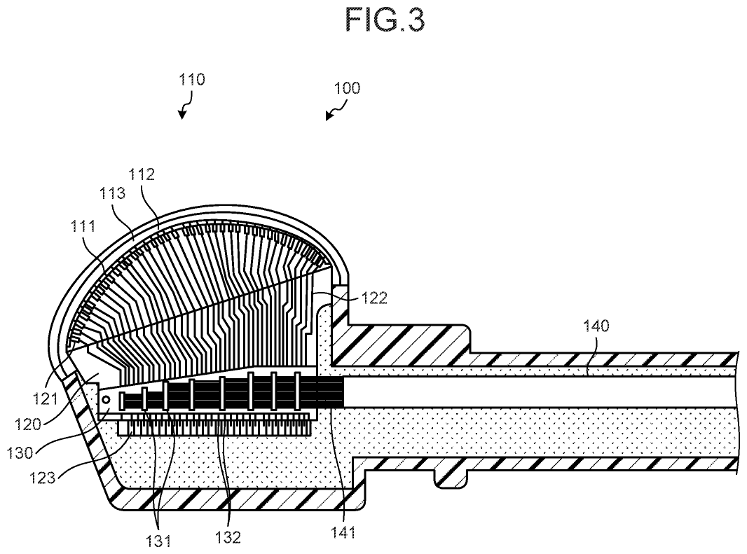

FIG. 3 is a cross-sectional view of the transducer unit in a longitudinal direction, which is used in the ultrasound diagnosis apparatus illustrated in FIG. 1. The transducer unit 100 includes an ultrasound transmission/reception unit 110, a transducer substrate 120, a cable substrate 130, and a coaxial cable bundle 140.

The ultrasound transmission/reception unit 110 includes, for example, a plurality of ultrasound transducers 111 each having a rectangular shape, electrode terminals (not illustrated) that are arranged on end portions of the ultrasound transducers 111, an acoustic matching layer 112, and an acoustic lens 113. Long sides of the plurality of ultrasound transducers 111 are connected to one another and form a convex-type transducer array that is arranged so as to be curved in a circular arc manner. The transducer array of the ultrasound transmission/reception unit 110 illustrated in FIG. 2 and illustrated in FIG. 3 is of a convex type, but, for example, it may be possible to adopt a radial type transducer group in which transducers are arranged in a two-dimensional array or a linear type transducer group that is not curved.

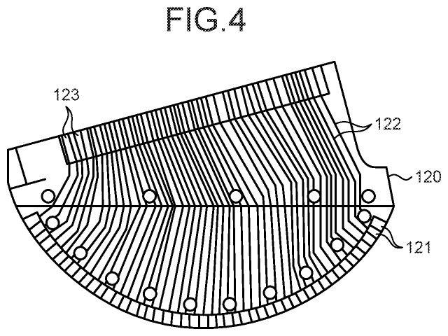

FIG. 4 is a plan view for explaining the transducer substrate. As illustrated in FIG. 4, the transducer substrate 120 includes a plurality of first electrode portions 121 that are electrically connected to the ultrasound transducers, a plurality of wiring patterns 122 that are connected to the first electrode portions 121, and a plurality of second electrode portions 123 that are arranged on other ends of the jwiring patterns 122. The transducer substrate 120 is a rigid substrate, such as a glass epoxy substrate. The first electrode portions 121 are electrically connected to the respective electrode terminals that are arranged on the end portions of the ultrasound transducers 111.

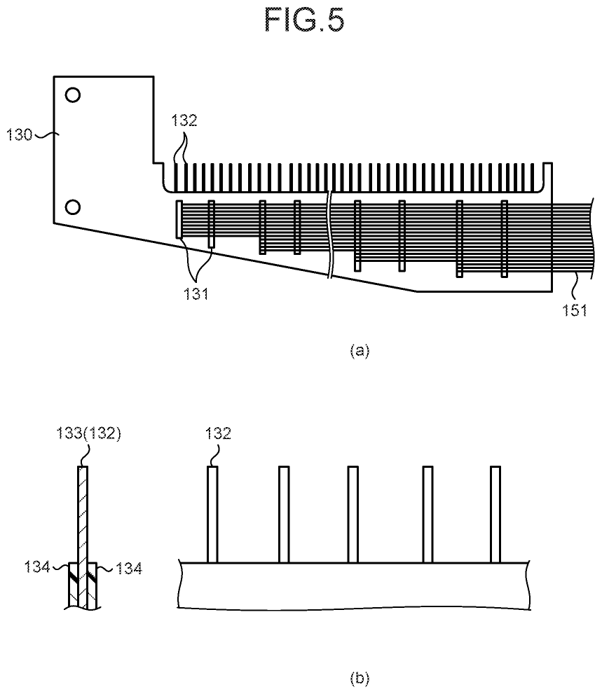

FIG. 5 is a plan view for explaining the cable substrate, where (a) of FIG. 5 is a plan view of the cable substrate and (b) of FIG. 5 is an enlarged plan view and a cross-sectional view of peripheries of flying leads. As illustrated in FIG. 5, the cable substrate 130 includes a plurality of electrode portions 131 and a plurality of flying leads 132. The plurality of flying leads 132 are arrayed in a direction perpendicular to an extending direction of the flying leads 132. The cable substrate 130 is a flexible printed circuit (FPC) board that includes a copper foil portion 133 that forms a wire pattern (not illustrated) and a polyimide layer 134 that covers both surfaces of the copper foil portion 133, and the flying leads 132 are formed by exposing the copper foil portion 133 from the polyimide layer 134. As illustrated in FIG. 3, a core wire and a shield of a coaxial wire 141 that is inserted in the coaxial cable bundle 140 is electrically connected to the electrode portions 131 by solder or the like (not illustrated), and the flying leads 132 are connected to the second electrode portions 123 of the transducer substrate 120.

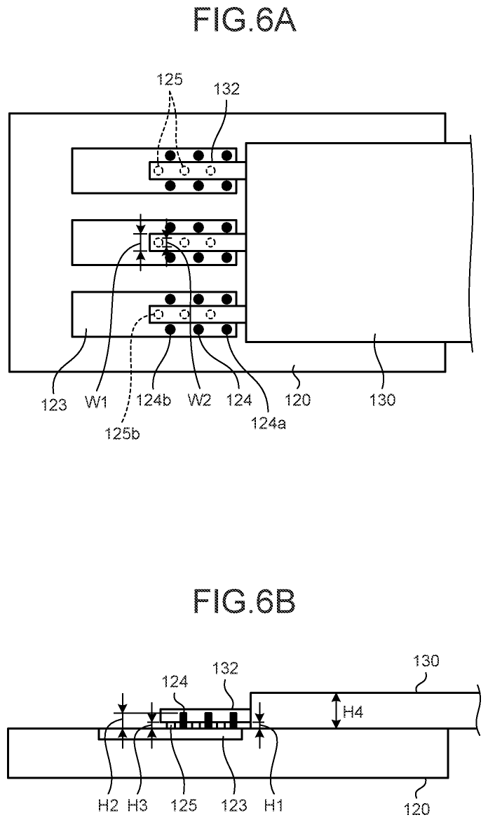

FIG. 6A is a partially enlarged plan view of connection portions of the transducer substrate and the cable substrate, and FIG. 6B is a partially enlarged side view of the connection portions of the transducer substrate and the cable substrate. In FIG. 6A and FIG. 6B, illustration of solder for connecting the flying leads 132 to the second electrode portions 123 is omitted. As illustrated in FIG. 6A and FIG. 6B, first protrusions 124 that determine positions of the flying leads 132 in a direction perpendicular to the extending direction of the flying leads 132 and second protrusions 125 on which the flying leads 132 are arranged are provided on the second electrode portions 123 to which the flying leads 132 are connected. An end portion of the cable substrate 130 in which the flying leads 132 are exposed is arranged so as to come into contact with the transducer substrate 120, and the polyimide layer serving as an exterior of the cable substrate 130 is fixed to the transducer substrate 120.

The first protrusions 124 are arranged in two rows along a horizontal direction (a vertical direction in the sheet of FIG. 6A) of each of the flying leads 132. The first protrusions 124 are arranged along the extending direction of the flying leads 132, so that it is possible to prevent the flying leads 132 bending in the horizontal direction when the flying leads 132 are heated and pressed so as to be connected to the second electrode portions 123, and it is possible to prevent occurrence of pattern deviation and disconnection of the flying leads 132.

It is preferable that a height H2 of each of the first protrusions 124 is larger than a distance H1 between each of the flying leads 132 and the transducer substrate 120 (a thickness of the polyimide layer 134 of the cable substrate 130) when the cable substrate 130 is arranged on the transducer substrate 120. By increasing the height H2 of each of the first protrusions 124 relative to the distance H2 between each of the flying leads 132 and the transducer substrate 120, it is possible to infallibly prevent the flying leads 132 from bending in the horizontal direction. Further, it is preferable that the height H2 of each of the first protrusions 124 is equal to or smaller than 1.5 times of a thickness H4 of the cable substrate 130. If the height H2 of each of the first protrusions 124 is equal to or smaller than 1.5 times of the thickness H4 of the cable substrate 130, it becomes easy to insert the flying leads 132 between the first protrusions 124.

The second protrusions 125 are arranged between the first protrusions 124 that are arranged in two rows, that is, in central portions of the second electrode portions 123 and in parallel to long-side directions of the second electrode portions 123. A width W2 of each of the second protrusions 125 is smaller than an interval W1 between the first protrusions 124 in the horizontal direction. By inserting the flying leads 132 between the first protrusions 124, the flying leads 132 are arranged on the second protrusions 125. By arranging the flying leads 132 on the second protrusions 125, it is possible to prevent the flying leads 132 from bending in a vertical direction (in a vertical direction in the sheet of FIG. 6B).

A height H3 of each of the second protrusions 125 is equal to or larger than 50% and equal to or smaller than 150% of the distance H1 between each of the flying leads 132 and the transducer substrate 120 when the cable substrate 130 is arranged on the transducer substrate 120, and more preferably, about the same as the distance H1. By setting the height H3 of each of the second protrusions 125 in the range as described above, it is possible to infallibly prevent the flying leads 132 from bending in the vertical direction.

As for arrangement positions of the first protrusions 124 and the second protrusions 125 that are arranged on the second electrode portions 123, it is preferable to arrange the first protrusions 124 and the second protrusions 125 such that the first protrusions 124 and the second protrusions 125 do not overlap with each other in the horizontal direction (in a horizontal direction in FIG. 6B and in the vertical direction in FIG. 6A). Furthermore, it is preferable that first protrusions 124a that are located on most proximal end sides of the flying leads 132, that is, at a side of the cable substrate 130, among the plurality of first protrusions 124 are arranged on exposed end portions on the proximal end sides of the flying leads 132. With this configuration, moment is less likely to be applied to the flying leads 132 in the horizontal direction (in the vertical direction in the sheet of FIG. 6A) at the time of soldering, so that it is possible to prevent disconnection. Furthermore, it is preferable that second protrusions 125b that are located on most distal end sides of the flying leads 132 among the plurality of second protrusions 125 are arranged on distal end sides relative to first protrusions 124b that are located on the most distal end sides of the flying leads 132 among the plurality of first protrusions 124.

The first protrusions 124 and the second protrusions 125 have cylindrical shapes, but embodiments are not limited to this example, and may have prism shapes (wall shapes). Further, in general, the cable substrate 130 that is used in the transducer unit 100 is configured such that the thickness of the polyimide layer 134 is set to 27.5 micrometers (μm) or 37.5 μm and a total thickness of the copper foil portion 133 and the polyimide layer 134 is set to 42.5 μm or 52.5 μm. It is more preferable that the height H2 of each of the first protrusions 124 when the cable substrate 130 as described above is used is set to 40 μm to 90 μm.

In the present embodiment, the first protrusions 124 and the second protrusions 125 are arranged on the second electrode portions 123 to which the flying leads 132 are connected, so that it is possible to effectively prevent deviation of the connection portions and disconnection of the flying leads 132.

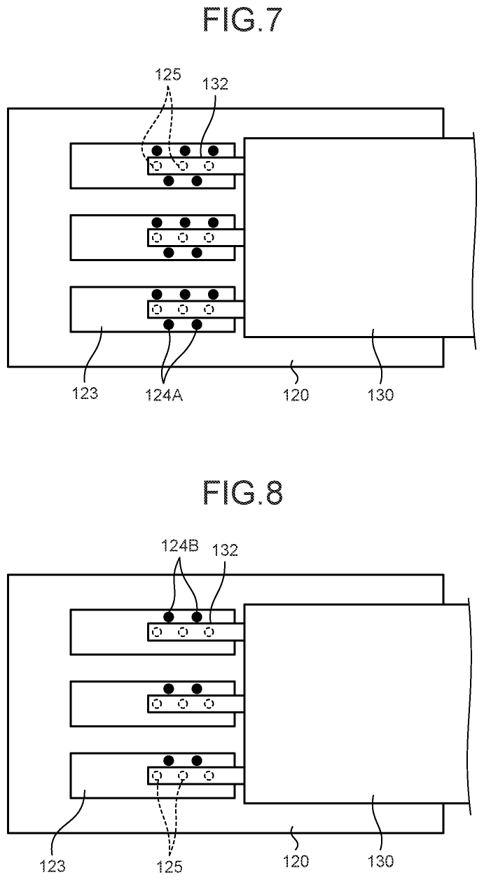

Meanwhile, in the embodiment as described above, the first protrusions 124 are arranged in a bilaterally symmetric manner (in a vertically symmetric manner in the sheet of FIG. 6A), but embodiments are not limited to this example, and, as illustrated in FIG. 7, first protrusions 124A may be arranged in a zig-zag manner. Further, it is preferable that the first protrusions 124 are arranged along both sides of each of the flying leads 132 as illustrated in FIG. 6A, but even if the first protrusions 124 are arranged on one side of each of the flying leads 132 as illustrated in FIG. 8, it is possible to prevent deviation of the connection portions and reduce bend of the flying leads 132 in the horizontal direction.

Furthermore, it is preferable to arrange the first protrusions 124 and the second protrusions 125 on the second electrode portions 123 to prevent bend of the flying leads 132; however, even if only the first protrusions 124 are arranged without arranging the second protrusions 125, it is possible to prevent the flying leads 132 from bending in the horizontal direction and reduce possibility of disconnection.

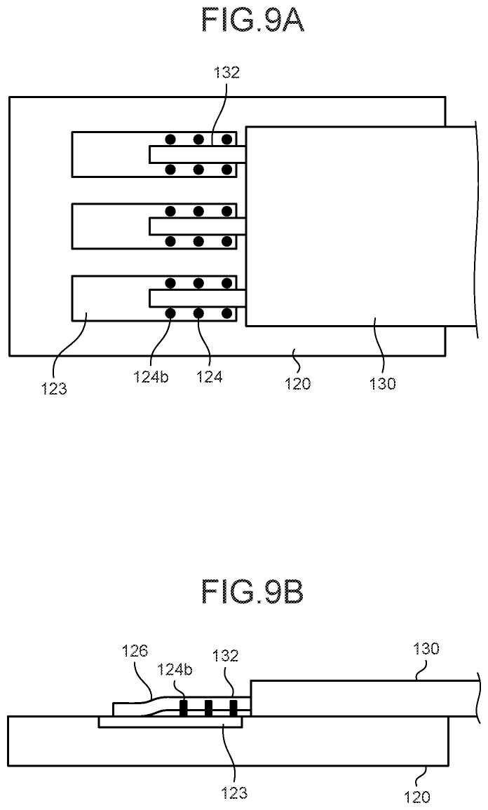

FIG. 9A is a partial enlarged plan view of connection portions of a transducer substrate and a cable substrate according to a third modification of the embodiment of the disclosure, and FIG. 9B is a partial enlarged side view of the connection portions of the transducer substrate and the cable substrate according to the third modification of the embodiment of the disclosure. In the third modification, only the first protrusions 124 are arranged on the second electrode portions 123 and the second protrusions 125 are not arranged. The second protrusions 125 are not arranged, and therefore, inclined portions 126 that bend in a vertical direction (in a vertical direction in the sheet of FIG. 9B) are formed in the flying leads 132 at the time of connection, but it is possible to prevent bend in the horizontal direction (in the vertical direction in the sheet of FIG. 9A) and reduce disconnection of the flying leads 132. Meanwhile, when only the first protrusions 124 are arranged on the second electrode portions 123, it is preferable to the first protrusions 124b, which are located on the most distal end sides of the flying leads 132 among the plurality of first protrusions 124, on the proximal end sides of the flying leads 132 relative to the inclined portions 126. With this configuration, it is possible to distribute stress that is applied to the connection portions of the flying leads 132 to the first protrusions 124, so that it is possible to prevent disconnection of the flying leads 132.

As described above, the substrate connection structure according to the disclosure is useful for a transducer unit in which a plurality of coaxial cables are connected to a cable substrate, and is particularly preferable for an ultrasound diagnosis apparatus that needs to be downsized.

The substrate connection structure according to the disclosure is able to prevent deviation between connection portions of an FPC board and a rigid substrate and reduce possibility of disconnection of flying leads, so that it is possible to provide a highly reliable ultrasound diagnosis apparatus.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A substrate connection structure comprising:
a rigid substrate that includes
a plurality of electrodes, and
a plurality of wiring patterns that are electrically connected to the plurality of electrodes;
a flexible printed board that includes a plurality of flying leads formed on one end of the flexible printed board, the flying leads being electrically connected to the plurality of electrodes; and
second protrusions that are arranged on the plurality of electrodes and on which the flying leads are arranged.

2. The substrate connection structure according to claim 1, wherein the rigid substrate further includes first protrusions that are arranged on the plurality of electrodes, the first protrusions being configured to determine positions of the flying leads in a direction perpendicular to an extending direction of the flying leads.

3. The substrate connection structure according to claim 1, wherein
an end portion of an insulator of the flexible printed board at a side at which the flying leads are exposed is in contact with the rigid substrate, and
the flying leads include, on distal end sides of the flying leads, inclined portions that are inclined in a direction toward the electrodes.

4. The substrate connection structure according to claim 3, wherein the first protrusions are arranged on proximal end sides of the flying leads relative to the inclined portions of the flying leads.

5. The substrate connection structure according to claim 4, wherein the first protrusions are arranged on exposed end portions of the flying leads on the proximal end sides of the flying leads.

6. The substrate connection structure according to claim 2, wherein the first protrusions are arranged on both sides of each of the flying leads.

7. The substrate connection structure according to claim 2, wherein a height of each of the first protrusions is larger than a distance between each of the flying leads and the substrate.

8. The substrate connection structure according to claim 7, wherein the height of each of the first protrusions is smaller than 1.5 times of a thickness of the flexible printed board.

9. The substrate connection structure according to claim 2, wherein the second protrusions are arranged on distal end sides of the flying leads relative to the first protrusions.

10. The substrate connection structure according to claim 1, wherein the plurality of the flying leads are arrayed in a direction perpendicular to an extending direction of the flying leads.

11. An ultrasound diagnosis apparatus comprising:
a plurality of ultrasound transducers each transmitting and receiving ultrasound waves;
a rigid substrate that includes
a plurality of electrodes, and
a plurality of wiring patterns that are electrically connected to the plurality of electrodes and the plurality of ultrasound transducers, the rigid substrate being fixed to the plurality of ultrasound transducers;
a flexible printed board that includes a plurality of flying leads formed on one end of the flexible printed board, the flying leads being electrically connected to the plurality of electrodes; and
second protrusions that are arranged on the plurality of electrodes and on which the flying leads are arranged.

12. The substrate connection structure according to claim 2, wherein a length of each of the first protrusions in the extending direction is smaller than a length of each of the flying leads in the extending direction.

13. The substrate connection structure according to claim 2, wherein the first protrusions are arranged along the extending direction.

14. The substrate connection structure according to claim 1, wherein a height of each of the second protrusions is equal to or larger than 50% and equal to or smaller than 150% of a distance between each of the flying leads and the substrate.

15. An ultrasound endoscope comprising:
an insertion portion configured to be inserted into a subject; and
an ultrasound transducer unit arranged on the insertion portion, the ultrasound transducer unit comprising:
a plurality of ultrasound transducers each transmitting and receiving ultrasound waves;
a rigid substrate that includes
a plurality of electrodes, and
a plurality of wiring patterns that are electrically connected to the plurality of electrodes and the plurality of ultrasound transducers, the rigid substrate being fixed to the plurality of ultrasound transducers;
a flexible printed board that includes a plurality of flying leads formed on one end of the flexible printed board, the flying leads being electrically connected to the plurality of electrodes; and
second protrusions that are arranged on the plurality of electrodes and on which the flying leads are arranged.
